(19) United States
(12) Patent Application Publication
(10) Pub. No.: US 2005/0089906 A1
(43) Pub. Date: Apr. 28, 2005

(54) Haplotype estimation method

(75) Inventors: Toshio Furuta, Tokyo (JP); Masao Yanagisawa, Tokyo (JP); Naoyuki Kamatani, Tokyo (JP)

Correspondence Address: OSTROLENK FABER GERB & SOFFEN, 1180 AVENUE OF THE AMERICAS, NEW YORK, NY, 100368403, US

(73) Assignee: NEC Corporation et al.

(21) Appl. No.: 10/943,299

(22) Filed: Sep. 17, 2004

(30) Foreign Application Priority Data
Sep. 19, 2003 (JP) 327943/2003

(51) Int. Cl. C12Q 1/68; G06F 19/00; G01N 33/48; G01N 33/50

(52) U.S. Cl. 435/6; 702/20

(57) ABSTRACT

An EM algorithm and a graph structure are combined so that all haplotype information to be assumed is kept, thus changing a problem into one for searching for a complete graph having a maximum score for haplotype estimation.

This application claims priority to prior application JP 2003-327943, the disclosure of which is incorporated herein by Reference.

### BACKGROUND OF THE INVENTION

The present invention relates to a haplotype estimation method which can be widely applied to research using genomic polymorphism markers. The term “research using genomic polymorphism markers” as used here means association study, tailor-made medicine, and so forth.

As widely known, the 3 billion chemical base pairs which make up the human genome have been determined and reported. In the sequence of bases in human genes (DNA base sequence), a variation between individuals in a population which occurs with a frequency of at least around 1% is called a genetic polymorphism. Genetic polymorphism is also simply called a polymorphism. Polymorphism is, in the individual genetic differences among the same breed, the difference defined by genes. Various levels of the polymorphism are present.

As also widely known, DNA (which is short for deoxyribonucleic acid) is almost entirely made up of the four bases of adenine (A), guanine (G), cytosine(C), and thymine (T).

Particularly, a polymorphism which exist regarding a single base is called a Single Nucleotide Polymorphism (SNP). SNPs are being given particular attention since they are polymorphisms which occur with a particularly high frequency. SNPs are estimated to exist at a rate of one every several hundred base pairs to 1,000 base pairs, and accordingly, 3 million to 10 million SNPs are assumed to exist in the human genome. Further, it is assumed that the great part of individual differences are determined by the differences in SNPs.

SNPs are thought to be easily employed as polymorphism markers, and development of equipment for clinical use is thought to be relatively easy, due to the following reasons.
(1) Several million copies or more exist within the genome.
(2) Determination is extremely easy, and results can be binarized, facilitating information processing.
(3) Technology for high-speed and massive-quantity SNP typing is in the process of being realized, and automated typing is not far away.

Hereinbelow the description will be made taking as an example a single nucleotide polymorphism to be used as a polymorphism marker. However, it is applicable to various other polymorphism markers such as a microsatellite.

Revealing the effects of SNPs on genetic functions and genetic manifestation should lead to discovery of predispositions susceptible to certain diseases, better medical treatment tailored to such predispositions, and enabling selection and development of drugs and pharmaceuticals accordingly.

Of all SNPs, a set of multiple SNPs existing in proximity on a gene, which have been extracted as a genetic function block, is referred to as a haplotype.

Now, with X and Y each representing one of the bases adenine (A), guanine (G), cytosine(C), and thymine (T), there are three patterns for an SNP at one base site, e.g., X/X, X/Y, and Y/Y.

There are primarily two approaches for genetic analysis, the functional approach and the genetic statistical approach. The former is an approach to analyze the functions of SNPs by molecular biology of the SNPs contained in genes. The latter is an approach to combine SNPs with clinical information and to extract the relation by genetic statistics. In the field of genetic statistics, methods for statistically uncovering potential relations between gene database and clinical databases are being studied. Genetic statistics allows the tendency of relations potentially existing in these database to be statistically uncovered.

The basis of genetic statistics is Mendel's Laws and linkage. It is crucial to understand Mendel's laws as laws pertaining to probability events. Mendel thought that there is behind the “phenotype” attributes of an individual organism which can be humanly recognized, a “genotype” which is not humanly recognizable, and that the “genotype” is stable only for the individual. He then discovered alleles (i.e., genes) which are stable across generations. The laws of Mendel can be understood to be laws relating to the above “phenotype”, “genotype”, and “alleles”. A locus is defined as a position where the three concepts can be defined. The laws of Mendel are summarized into the later-described “the law of segregation”, “the law of dominance”, and “the law of independent assortment”.

The nature observed at the level of individuals is expressed in terms of phenotype and trait. Traits are categories, and phenotypes are individual types observed in the traits., The concepts of locus, allele, and genotype must be defined along the line of Mendel in order to analyze the relation of individual traits.

A great number of loci are thought to exist for one mating population, generally stable in a species. The genotype is a stable unit for an individual. Each individual has one genotype at each locus. From a chromosomal Expression perspective, a locus is a portion between minimal units where recombination occurs. From a molecular biology perspective, a locus is a single base. A locus is also referred to as a genetic locus.

While a genotype is stable on the individual level, a genotype has no stability across generations. Alleles are handed down in a stable manner from individual to individual, not genotypes. An allele is also called an allelomorph.

Mendel's law of segregation is a law regarding components making up genotypes which are stable while traversing generations. This law of segregation asserts that “a genotype is a combination of two alleles, and only one of the two alleles making up the genotype will be passed on to the next generation, at a probability which is equal to that of the other allele being passed on”. In other words, this law deals with probability. Accordingly, an allele is the smallest unit which can be handed from one generation to another in a stable manner. Of course, mutations destroy alleles, but Mendel's laws do not take mutations into consideration. At the chromosomal level, an allele is a locus on one strand of the chromosome, and in molecular biology is a type on one strand of the chromosome such as an SNP, STRP (short tandem repeat polymorphism), VNTR (variable number of tandem repeat), and so forth. For example, in an SNP, this would be one side of the base pair, such as T (thymine) or C (cytosine).

Mendel's law of domination asserts that “there are laws relating to the correlation between genotype and phenotype, and the phenotype of an individual is a function of the genotype”, indicating that there is dominance and non-dominance (i.e., recessive) in the function. At the chromosomal level, a genotype is a combination of two homologous loci existing at a homologous site on two homologous chromosomes. At the molecular biology level, a genotype is a combination of a polymorphism such as an SNP, or an STRP, a VNTR, or the like.

Linkage is an exception to Mendel's law of independent assortment. Mendel's law of independent assortment is a law relating to the relation between multiple loci, and asserts that “alleles at different loci are distributed to the child independently from one another”. At the chromosomal level, this is correct only regarding loci on different chromosomes. Two alleles on the same chromosome are physically joined, and accordingly enter the gamete together and are passed on to the child, meaning that the law of independent assortment is inapplicable. However, alleles on the same chromosome may not remain completely joined on to the next generation in some cases, since rearrangement of the joined alleles can occur due to crossing of the chromosome at the time of meiosis (recombination). The greater the distance between the loci is, the greatest the probability of recombination.

Morgan showed that Mendel's law of independent assortment does not necessarily apply to genes with different loci, thereby discovering linkage. However, the law of independent assortment is still correct regarding loci on different chromosomes, and moreover, it is crucial to understand that the law of independent assortment is axiomatic to the concept of linkage. Accordingly, the idea that Mendel's law of independent assortment was a mistake, is incorrect.

Now, the term independent means that the probability of two phenomena occurring simultaneously is expressed by the product of the probabilities of each phenomenon occurring individually. In genetic statistics, the term independent assortment does not mean being on the same chromosome, but rather is concerned with probability and statistics.

Defining linkage allows definition of combinations of the alleles at Locus 1 and Locus 2 (haplotype). A haplotype is a combination of alleles at linked loci in one gamete. A haplotype is preserved unless recombination occurs in meiosis. Recombination forms a new haplotype, which is passed on to the next generation by the gamete, and remains unchanged until recombination occurs again. The existence of a haplotype is a function of the haplotype in the gamete passed on from the parent and whether or not recombination has occurred between loci. The phenomenon of recombination occurs according to probability following the rate of recombination, so haplotype heredity can be thought of as a probability phenomenon having the recombination rate as a probability distribution function thereof. Linkage analysis calculates the order and distance of loci optimal for observation data relating to traits and genotypes of a lineage.

Each individual has two haplotypes, and the combination of the haplotypes is also referred to as a diplotype configuration.

On the assumption that one lineage has transmission of n gametes and m loci in linkage, consideration will herein be made about m inheritance vectors which represent the recombination event in the meiosis occurred in the lineage. One inheritance vector corresponds to one locus and is a column vector having n factors. Each factor corresponds to each of the meioses consecutively numbered. The factor of the inheritance vector is either 1 or 0. 0 represents the fact that the individual in which the meiosis occurred transmits the allele, inherited from the father, to the child by the gametes. 1 represents the fact that the allele, inherited from the mother, is transmitted to the child by the gametes. Thus, when represented by the inheritance vector, the essence resides in the fact about which allele have been transmitted to the child.

Generally, permutation genotypes of all the members of the lineage can be determined by permutation genotypes and the inheritance vectors of all the founders. The permutation genotype is a combination of two alleles with the information indicative of the origin of the allele, i.e., which allele belongs to the father-origin (i.e., the permutation). The genotypes 1/2 and 2/1 are different permutation genotypes. Generally, the genotype is a combination of alleles, not the permutation of the alleles (the combination genotypes).

The concept of linkage disequilibrium is paramount in genetic statistics. The difference between linkage and linkage disequilibrium must be correctly understood. The concept of linkage has been described above as an exception to Mendel's law of independent assortment, and that independent assortment is purely a concept of probabilistic concern. True linkage disequilibrium is a concept based on the assumption of linkage.

Genotypes exist at multiple linked loci, and if sufficient lineage information can be obtained, the diplotype configuration of the individual (the combination of two haplotypes) can be determined, as described above. A haplotype is a combination of individual alleles at loci on the same chromosome (i.e., linked). In case where lineage information does not exist, all information regarding the genetic information of the individual is the diplotype configuration of the individual.

Linkage disequilibrium is a law regarding distribution of haplotypes in a population. A diplotype configuration is a combination of two haplotypes. Therefore, the number of haplotypes in a population is double the number of individuals in that population.

Linkage disequilibrium is a phenomenon observed at two or more linked loci. In simple terms, linkage disequilibrium is a phenomenon wherein distribution of alleles between different loci is not independent, i.e., wherein the haplotype frequency deviates from the frequency obtained by multiplying the frequencies of alleles at the loci. Conversely, a state wherein the haplotype frequency equals the product of allele frequency (i.e., independent), is referred to as linkage equilibrium, and therefore, a state wherein linkage equilibrium is not realized is a state of linkage disequilibrium.

If a population is sufficiently great, and sufficient time has passed since all mutations have occurred, and further there is no change in the allele frequency, linkage equilibrium can be expected. However, in reality, no population is large enough, sufficient time has not passed, and moreover, the allele frequency changes. Further, in case where there is mixing between two populations which have each attained linkage equilibrium, strong linkage disequilibrium can be expected. Generally, almost all populations in the world exhibit strong linkage disequilibrium at close genetic distances. The farther the polymorphism, the smaller the degree of linkage disequilibrium is. Haplotypes involving new mutations are expected to exhibit the strongest linkage disequilibrium, and the greater the effective size of the population is, the smaller the linkage disequilibrium is expected to be.

The term “genetic (map) distance” is defined as “the value expected for the number of times of crossing between two loci”. Specifically, genetic distance is a concept defined based on cross-over, rather than recombination. The unit of measurement is Morgan (M). It should be understood however, that one cross-over does not necessarily occur per 1 M, rather, this is a concept of probability. Also, cross-over is a phenomenon in which partial crossing over occurs among homologous chromosomes during meiosis. Cross-over is a chromosomal event which can be observed through the microscope, and refers to the action of crossing over. The number of times of cross-over per meiosis may not necessarily be one.

Linkage disequilibrium provides genetic statistics with many techniques. If the prediction of common-disease/common-variant/common-origin is correct, there should be a strong linkage disequilibrium between variations in disorders and variations in the surroundings, and linkage disequilibrium should be able to be used to find disorder-related sites.

Regions with strong linkage disequilibrium exist between regions where recombination has historically occurred. These regions are referred to as blocks, and are said to be around 10 to 15 kb (kilobase pair) in physical distance, though it is known that there are occasionally blocks around 100 kb or so. It is noted here that physical distance refers to the physical distance between two loci upon reading the base sequence.

In association studies, normally, polymorphism markers are disposed at equal intervals. “Association studies” as used here means case-control study or patient-oriented study, which is the easiest sort of study. As with linkage disequilibrium studies, association studies also handle populations of individuals with no lineage data. Samples of a patient population and a general population are collected separately, and difference in allele frequency therebetween, or difference in the frequency of individuals having particular alleles, is studied. This means that a greater number of markers are placed in a 100 kb block as compared to blocks of around 10 to 15 kb. For example, 30 or more SNPs may be typed for a massive block of 100 kb or more.

However, the cumulative frequency reaches or exceeds 95% with 5 or less haplotypes in the block region. Therefore, the number of SNPs needed for one block is at least three, from 2²=4 and 2³=8. There is absolutely no need for 30 SNPs in order to identify a mere five haplotypes at the most. Accordingly, this is a waste.

SNPs (single nucleotide polymorphisms) are distributed in the human genome every 250 to 350 bp (e.g., Reference 1: L. Beaudet et al., “Homogenous assays for single-nucleotide polymorphism typing using AlphaScreen,” Genome Res., Vol. 11, pp. 600-608, 2001). SNPs with minor allele frequency of 0.1 or higher exist every 600 kb or so (e.g., Reference 2: D. G. Wang et al., “Large scale identification, mapping and genotyping of single-nucleotide polymorphisms in the human genome,” Science, vol. 280, pp. 1077-1082, 1998). SNPs have a low mutation rate and enable high-throughput typing, and therefore, are more effective than micro-satellite markers. Further, SNP haplotype information is extremely effective regarding testing for linkage disequilibrium and mapping disorder genes to chromosomes (e.g., Reference 3: S. E. Hodge, M. Boehnke and M. A. Spance, “Loss of information due to ambigunous haplotyping of SNPs,” Nat.Genet., vo.21, pp.360-361, 1999, Reference 4: M. J. Rieder, S. L. Taylor; A. G. Clark and D. A. Nickerson, “Sequene variation in the human angiotensin converting ensyme,” Nat.Genet., vol. 22, pp.59-62, 1999, Reference 5: N. Risch and K. Menrkangas, “The future of genetics studies of complex human diseases,” Science, vol.273, pp. 1516-1517, 1996). SNP haplotype information allows estimation of positional information of linkage disequilibrium (LD) in a more robust and powerful manner than single SNPs (e.g., Reference 6: M. J. Daly et al., “High-resolution haplotype structure in the human genome,” Nat.Genet., vol. 29, pp. 229-232, 2001, Reference 7: J. K. Pritchard, “Are rare variants responsible for susceptibility to compled diseases?,” Ann. Hum. Genet., vol.69, pp.124-137).

Examples of techniques for estimating haplotypes on the molecular level include single-molecular dilution (e.g., Reference 8: G. Ruano, K. K. Kidd and J. C. Stephens, “Haplotype of multiple polymorphisms resolved by enzymatic amplification of single DNA molecules,” in Proc. Natl. Acad. Sci. USA, vol. 87, pp.6296-6300, 1990), long-range PCR (e.g., Reference 9: S. Michalatos-Beloin et al., “Molecular haplotyping of genetic markers 10 kb apart by allele-specific long-range PCR,” Nucleic Acids Res., vol.24, pp.4841-4843, 1996), isothermal rolling-circle amplification, (e.g., Reference 10: P M. Lizardi et al., “Mutation detection and single-molecule counting using isothermal rolling-circle amplification,” Nat. Genet., vol. 19, pp. 225-232, 1998), and conversion from diploid chromosomes to haploid chromosomes (e.g., Reference 11: J. A. Dougles et al., “Expermentally-derived haplotypes substantially increase the efficiency of linkage disequilibrium studies,” Nat. Genet., vol. 28, pp. 361-364, 2001). However, these techniques have many problems such as difficulty in automation, high costs, low-throughput, and so forth.

Therefore, attention has been given to techniques for statistically estimating haplotypes instead of on the molecular level. In case where lineage data is available, examples of techniques for determining the phase or haplotype include software such as Linkage Package (e.g., Reference 12: G. M. Lathrop et al., “Multilocus linkage analysis in humans: detection of linkage and estimation of recombination,” Am. J. Hum. Genet., vol. 37, pp.482-498, 1985) and GENEHUNTER (e.g., Reference 13: L. Kruglyak, M. J. Daly, M. P. Reeve-Daly and E. S. Lander, “Parametric and nonparametric linkage analysis: a unified multipoint approach,” Am. J. Hum. Genet., vol.58, pp.1347-1363, 1996), and techniques for estimating minimum recombinant haplotypes in a lineage based on several rules (e.g., Reference 14: M. Qian and L. Beckmann, “Minimum-Recombinant haplotyping in pedigrees,” Am. J. Hum. Genet., vol.70, pp.1434-1445, 2002).

On the other hand, in case where no lineage data exists, examples of techniques include Clark's algorithm (e.g., Reference 15: A. G. Clark, “Inference of haplotypes form PCR-amplified samples of diploid populations,” Mol Biol Evol, vol. 7, pp.111-122, 1990), the maximum-likelihood method (e.g., Reference 18: M. N. Chiano and D. G. Clayton, “Fine genetic mapping using haplotype analysis and the missing problem,” Ann. Hum. Genet., vol. 62, pp. 55-60, 1998, Reference 19: L. Excoffer and M. Slatkin, “Maximum-likelihood estimation of molecular haplotype frequencies in a diploid polulation,” Mol. Biol. Evol. vol. 12, pp.921-927, 1995, Reference 20: M. Hawley and K. Kidd, “HAPLO: a program using the EM algorithm to estimate the frequencies of multi-cite haplotypes,” J. Hered., vol.86, pp.409-411, 1995, Reference 21: T. Ito et al, “Estimation of haplotype frequencies, linkage-disequilibrium measures, and combination of haplotype copies in each pool by use of pooled DNA data,” Am, J. Hum, Genet., vol. 72, pp. 384-398, 2003, Reference 22: Y. Kitamura et al., “Detennination of probability distribution of diplotype configuration (diplotype distribution) for each subject from genotypic data using the EM algorithm,” Ann, Hum. Genet., vol. 66, pp.183-193, 2002, Reference 23: J. C. Long, R. C. Williams and M. Urbanek, “An E-M algorithm and testing strategy for multiple locus haplotytes,” Am. J. Hum. Genet., vol.56, pp.799-810, 1995) using the Expectation/Maximization (EM) algorithm (e.g., Reference 17: A. P Dempster, N. M. Laird and D. B. Rubin, “Maximum likelihood from incomplete data via the EM algorithm,” J. Roy. Statist. Soc. Ser. B, vol. 39, pp.1-38, 1977) by counting genes (e.g., Reference 16: J. Ott, “Counting methods (EM algorithm) in human pedigree analysis: linkage and segregation analysis,” Ann. Hum. Genet., vol.40, pp.443-454, 1977) under the assumption of Hardy Weinberg equilibrium (HWE), the pseudo Gibbs Sampler (e.g., Reference 24: M. Stephens, N. J. Smith and P. Donnely “A new statistical method for haplotype reconstruction from population data,” Am. J. Hum. Genet., vol. 68, pp.978-989, 2001), the PL (Partition-Ligation) method (e.g., Reference 25: T. Niu, Z. S. Qin, X. Xu and J. S. Liu, Baysian haplotype inference for multiple linked single nucleotide polymorphisms,” Am. J. Hum. Genet., vol.70, pp.157-169, 2002), and so forth.

Estimation using the EM algorithm is considered to be the most precise, and is currently mainstream.

In detail, estimation using the EM algorithm is a technique in which an appropriate value is set for an initial value of haplotype frequency, HWE is assumed in the E-step to obtain the expected value of the haplotype, and the haplotype frequency is updated in the M-step, which is repeated to obtain the maximum-likelihood estimation value for the haplotype frequency, and this technique is currently the most popular.

In Reference 22, Kitamura et al. propose a technique using the haplotype frequency of a population to determine the haplotype of individuals therein using the Bayes method. In Reference 21, Ito et al. propose a technique using genotypes obtained from a DNA pool containing the DNA of multiple individuals to estimate the combination of haplotype copies in the DNA pool and the haplotype frequency of the population.

The EM algorithm depends upon the HWE for the E step. However, Fallin et al. evaluated haplotype estimation frequencies with the EM algorithm under the Hardy Weinberg disequilibrium (HWD), and note that while the estimation precision drops due to the HWD, the estimation precision increases due to increased homozygous degree, and therefore, there is a balance between the two (e.g., Reference 26: D. Fallin and N. J. Schork, “Accuracy of haplotype frequency estimation for biallelic loci, via the expectation-maximization algodthm for unphased diploid genotype data,” Am. J. Hum. Genet., vol.67, pp.947-959, 2000).

Techniques using the EM algorithm are currently mainstream because various types of data can be estimated with high precision. However, all haplotype frequency information which can be assume must be kept. It is therefore difficult to estimate multilocus genotype data because of the amount of calculations.

In order to solve such a problem, in Reference 24, Stephens et al have proposed a Phase method using the PGS (pseudo Gibbs Sampler). This is a Gibbs sampler combining an improved Clark's algorithm and a simulation based on a coalescence model, and has precision equal to or greater than that of the EM algorithm while being capable of handling even more gene loci.

Zhang et al. have suggested that the number of markers can be reduced by using tag SNPs representing haplotypes. (e.g., Reference 27: K. Zhang, P. Calabrace, M. nordborg and F. Sun, “Haplotype block structure and its applications to association studies: power and stufy designs,” Am. J. Hum. Genet., vol. 71, pp. 1386-1394, 2002)

Considering genome-wide haplotypes, it is known that the human genome can be divided into independent haplotype blocks (e.g., Reference 6, Reference 28: E. Dawson et al., “A first-generation linkage disequilibdum map of human chromosome 22,” Nature, vol. 418, pp.544-548, 2002, Reference 29: S. B. Gabriel et al, “The structure of haplotype blocks in the human genome,” Science, vol. 296, pp.2225-2229, 2002, and Reference 30: G. C. Johnson et al., “Haplotype tagging for the identification of common disease genes,” Nat. Genet., vol.29, pp.233-237, 2001).

In Reference 6, Daly et al. analyzed 103 SNPs having a minor allele frequency of 5% or more within a range of 500 kb of the human chromosome 5p31 which is thought to be related to Crohn's disease, and found that this range can be divided into 11 blocks. Further, in Reference 30, Johnson et al. analyzed 122 SNPs in a 135 kb range of nine genes, and concluded that 34 SNPs were sufficient to characterize the haplotypes of 384 Europeans. In Reference 25, Niu et al. have succeeded in handling a greater number of gene loci by the use of a technique called PL (Partition-Ligation) in which gene loci are sectioned into haplotype blocks and haplotypes are studied for each block.

Now, linkage disequilibrium never exceeds an average of 3 kb in a general population, and it is believed that 500,000 or more SNPs will be necessary for association study using the entire genome (e.g., Reference 31: L. Kruglyak, “Prospects for whole-genome linkage disequilibrium mapping of complex disease genes,” Nat. Genet., vol. 22, pp.139-144, 1999).

Moreover, SNP data regarding IBD (Identity by descent) is known for the region of the human chromosome 5p31 (e.g., Reference 32: URL: http://www-genome.wi.mit.edu/human/IDB5/). It is noted here that IBD refers to a state in which two individuals carry the same allelic gene as an ancestor (i.e., homoeologous).

On the other hand, proposal has been made of a haplotype estimation algorithm in which a combination of genetic statistic technique using the EM algorithm and the issue of maximization of edge-weighting in a Markov model (e.g., Reference 33: Shimosato et al., “A New Haplotype Estimation Algorithm Applicable to Many SNPs Inputs,” FIT (Forum of Information Technology, 2002, A-15, pp.29-30). This proposed technique comprises two processes, a first process obtaining the probability of transition by using the EM algorithm between alleles at two arbitrary loci other than the current locus from input data, and a second process for creating a Markov model from the genotype data of the patient with the alleles of each loci as nodes, and estimating the most likely haplotype for the patient. In other words, this represents the diplotypes of an individual as a graph structure with the alleles as nodes.

In addition, a haplotype estimation technique capable of handling genotype data of a greater number of gene loci by division according to haplotype blocks and maximization of edge-weighting in a Markov model has been proposed (e.g., Reference 34: Shimosato et al, “A Proposal of Haplotype estimation for Many SNPs Inputs Using Block Division,” 2003, IEICE Gen. Conf. D-12-41, pp. 202). This technique is the same as that in Reference 33 except for dividing the haplotype blocks.

As described above, estimation using the EM algorithm is considered to be the most precise and is mainstream nowadays. However, the EM algorithm has the following problems. Specifically, all haplotypes which may exist within a population must be kept and all taken into consideration upon repetitive calculation. Therefore, the amount of calculations increases by O(2ⁿ) as to n loci. If n<30 holds, a calculator will be able to execute the calculations, but after exceeding n>30, the current haplotype estimation by EM algorithm becomes difficult to carry out by using a calculator. That is, the EM algorithm holds all haplotypes which a population can assume. Therefore, if gene loci increase, both execution time and memory usage problematically and exponentially are increased.

As mentioned above, it is believed that 500,000 or more SNPs will be necessary for association study using the entire genome. Therefore, genome-wide haplotype estimation using the EM algorithm is impossible.

In other words, increased demand for genome analysis and improved technology such as sequences will increase the scale of analysis, and the amount of data to be processed at one time will increase. Such increased data amount will become too great to handle for techniques in which each haplotype within a population has some sort of value as with the MCMC (Markov Chain Monte Carlo) method or EM algorithm in order to analyze data in which the number of loci n exceeds 30. As a consequence, it is difficult to execute on a calculator.

Further, as described above, using the PL method to section gene loci into haplotype blocks and study the haplotypes for each block has been proposed. However, recently, a clear division method for the haplotype blocks has not been established yet, and an algorithm for dividing into suitable blocks needs to be developed.

Moreover, according to the techniques proposed in Reference 33 and Reference 34, alleles are used as nodes (vertices). Therefore, it is difficult handle diplotypes for each individual in an integrated manner.

### SUMMARY OF THE INVENTION

It is therefore an object of the present invention to provide a haplotype estimation method which is capable of handling genotype data having a great number of loci n.

It is another object of the present invention to provide a haplotype estimation method which is capable of handling diplotype configurations for each individual in an integrated manner.

The present Inventors have proposed a new algorithm (haplotype estimation method) which improves the problems which many haplotype estimation methods such as the EM algorithm have with regard to the amount of calculations. According to the proposed technique, the EM algorithm and a graph structure are combined so that all haplotype information to be assumed are kept, thus changing the problem into one for searching for a complete graph having a maximum score for haplotype estimation.

Specifically, according to a first aspect of the present invention, a method for estimating diplotype configurations of each individual from genotype data with n loci, using a computer system, comprises: a step for representing diplotype configurations for each individual as an n-locus complete graph structure with multiple permutation genotypes corresponding to the vertices; a step for taking the weight of each edge of the n-locus complete graph structure as probability of diplotype configurations estimated by maximum-likelihood estimation from the genotype data; a step for selecting a set of vertices of the no locus complete graph structure using a predetermined score; and a step for estimating diplotype configurations of each individual by solving an n-node complete graph having a maximal score.

In the above method for estimating diplotype configurations, the EM algorithm may be used for maximum-likelihood estimation. The method may further comprise a step for handling individuals with unknown values in the genotype data by estimating the diplotype configuration excluding the unknown values, and then performing complementation using the results.

According to a second aspect of the present invention, a method for estimating haplotypes from genotype data with n loci, using a computer system, comprises: a step for representing diplotype configurations for each individual as an n-locus complete graph structure with multiple permutation genotypes corresponding to the vertices; a step for taking the weight of each edge of the n-locus complete graph structure as probability of diplotype configurations estimated by maximum-likelihood estimation from the genotype data; a step for selecting a set of vertices of the n-locus complete graph structure using a predetermined score; a step for estimating diplotype configurations of each individual by solving an n-node complete graph having a maximal score; and a step for obtaining haplotype frequency of the population from the diplotype configuration of each individual obtained by the estimation.

In the above method for estimating haplotypes, the EM algorithm may be used for maximum-likelihood estimation. The method may further comprise a step for handling individuals with unknown values in the genotype data by estimating the diplotype configuration excluding the unknown values, and then performing complementation using the results.

According to a third aspect of the present invention, a device for estimating diplotype configurations of each individual from genotype data with n loci, comprises: means for representing diplotype configurations for each individual as an n-locus complete graph structure with multiple permutation genotypes corresponding to the vertices; means for taking the weight of each edge of the n-locus complete graph structure as probability of diplotype configurations estimated by maximum-likelihood estimation from the genotype data; means for selecting a set of vertices of the n-locus complete graph structure using a predetermined score; and means for estimating diplotype configurations of each individual by solving an n-node complete graph having a maximal score.

In the above device for estimating diplotype configurations, the EM algorithm may be used for maximum-likelihood estimation. The method may further comprise means for handling individuals with unknown values in the genotype data by estimating the diplotype configuration excluding the unknown values, and then performing complementation using the results.

According to a fourth aspect of the present invention, a device for estimating haplotypes from genotype data with n loci comprises: means for representing diplotype configurations for each individual as an n-locus complete graph structure with multiple permutation genotypes corresponding to the vertices; means for taking the weight of each edge of the n-locus complete graph structure as probability of diplotype configurations estimated by maximum-likelihood estimation from the genotype data; means for selecting a set of vertices of the n-locus complete graph structure using a predetermined score; means for estimating diplotype configurations of each individual by solving an n-node complete graph having a maximal score; and means for obtaining haplotype frequency of the population from the diplotype configuration of each individual obtained by the estimation.

In the above device for estimating haplotypes, the EM algorithm may be used for maximum-likelihood estimation. The device may further comprise means for handling individuals with unknown values in the genotype data by estimating the diplotype configuration excluding the unknown values, and then performing complementation using the results.

According to a fifth aspect of the present invention, a program for causing a computer to estimate diplotype configurations of each individual from genotype data with n loci, comprises: a function for representing diplotype configurations for each individual as an n-locus complete graph structure with multiple permutation genotypes corresponding to the vertices; a function for taking the weight of each edge of the n-locus complete graph structure as probability of diplotype configurations estimated by maximum-likelihood estimation from the genotype data; a function for selecting a set of vertices of the n-locus complete graph structure using a predetermined score; and a function for estimating diplotype configurations of each individual by solving an n-node complete graph having a maximal score.

In the above program, the EM algorithm may be used for maximum-likelihood estimation. The program may further comprise a function for handling individuals with unknown values in the genotype data by causing a computer to estimate the diplotype configuration excluding the unknown values, and then performing complementation using the results.

According to a sixth aspect of the present invention, a program for causing a computer to estimate haplotypes from genotype data with n loci, comprises: a function for representing diplotype configurations for each individual as an n-locus complete graph structure with multiple permutation genotypes corresponding to the vertices; a function for taking the weight of each edge of the n-locus complete graph structure as probability of diplotype configurations estimated by maximum-likelihood estimation from the genotype data; a function for selecting a set of vertices of the n-locus complete graph structure using a predetermined score; a function for estimating diplotype configurations of each individual by solving an n-node complete graph having a maximal score; and a function for obtaining haplotype frequency of the population from the diplotype configuration of each individual obtained by the estimation.

In the above program, the EM algorithm may be used for maximum-likelihood estimation. The program may further comprise a function for handling individuals with unknown values in the genotype data by causing a computer to estimate the diplotype configuration excluding the unknown values, and then performing complementation using the results.

According to the present invention, the high precision of estimation of the EM algorithm and the short data processing time thereof for small amounts of data can be applied to large amounts of data so as to reduce the processing time from that of the order of exponential functions to that of the order of polynomials, Further, permutation loci are used instead of alleles. Therefore, diplotype configurations for each individual can be handled in an integrated manner.

### BRIEF DESCRIPTION OF THE DRAWINGS

FIG. 1 is a diagram illustrating a data structure of a graph;

FIG. 2 is a diagram illustrating an example of selecting one 4-node complete graph structure from a 4-locus data structure;

FIG. 3 is a diagram illustrating an algorithm for extracting an n-node complete graph structure in which sum of weight of edges is maximal, from an n-locus complete graph structure for estimating diplotype configurations of individuals;

FIG. 4 is a diagram describing an example of estimating unknown portions by compensation;

FIG. 5 is a diagram illustrating results of measuring executing time using SNP data relating to IBD in human chromosome 5q31 region;

FIG. 6 is a diagram illustrating an experiment for comparing the haplotype estimation method according to the present invention and the conventional method regarding time for executing simulation data, in which 100 individuals are involved and the number of loci is varied from 5 to 50;

FIG. 7 is a diagram illustrating an experiment for comparing the haplotype estimation method according to the present invention and the conventional method regarding estimation precision for executing simulation data in terms of mean error rate, in which 100 individuals are involved and the number of loci is varied from 2 to 16 in steps of 2;

FIG. 8 is a block diagram illustrating a hardware configuration of a computer system for realizing haplotype estimation method according to the present invention; and

FIG. 9 is a flowchart for describing a program for executing haplotype estimation method according to the present invention with a computer system shown in FIG. 8.

### DESCRIPTION OF PREFERRED EMBODIMENT

Detailed description will be made below regarding a haplotype estimating method according to the present invention.

The EM algorithm allows the researcher to perform high-precision haplotype estimation with a high speed for the data including a small number of loci. The haplotype estimating method according to the present invention has a graph structure employing the EM algorithm, thereby reducing time-calculations and space-calculations while maintaining the advantages of the EM algorithm, i.e., maintaining generally the same estimation precision. The haplotype estimating method according to the present invention will be referred to as “idlight” hereafter.

In general, the conventional algorithm, employing such a method in which all the conceivable haplotypes in the population are estimated, cannot handle genotype data including the n (more than thirty) loci due to explosively increased memory amount required for handling such data.

In order to resolve such problems, a new data structure needs to be proposed for handling the information with regard to all the haplotypes without holding the information with regard to the individual haplotypes.

In general, linkage disequilibrium occurs between each pair of the loci. In some cases, great linkage disequilibrium occurs not only between the loci adjacent one to another, but also between loci away one from another. Accordingly, there is the need to estimate the haplotypes giving consideration to the linkage disequilibrium occurring between all the locus pairs.

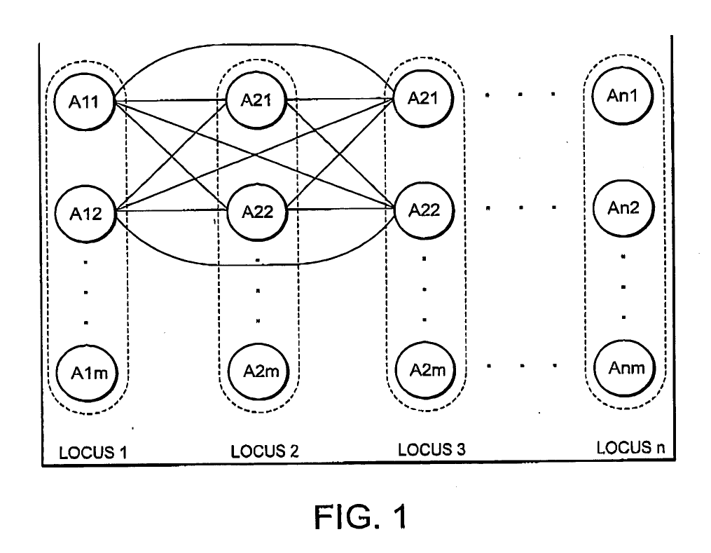

In general, multiple kinds of alleles can exist at each locus in the population. Therefore, the data structure must be estimated giving consideration to the linkage disequilibrium occurring between the alleles at the loci different one from another. Accordingly, the data structure including vertices (nodes) each of which corresponds to a single allele at a locus, and edges each of which are formed between the vertices different one from another, as shown in FIG. 1, has been proposed as an effective method for estimating the haplotypes in the population.

In this case, with the number of the loci as n, the haplotype data of the population can be handled as an n-locus complete graph which is a new data structure. The n-locus complete graph is represented by Kₘ_{ᵢ}ⁿ.

Here, mi represents the number of the alleles at the loci i. With the n-locus complete graph, the conceivable alleles at each locus are arrayed at the corresponding vertex, and each edge between the vertices is weighted by the value which exhibits the strength of connection between the corresponding vertices. The haplotype frequency between the corresponding two loci calculated in the population using the EM algorithm is employed as the aforementioned strength of connection. That is to say, the strength of connection of the alleles between the two loci must be estimated with giving consideration to the ratio of the alleles in the population, and the magnitude of the linkage disequilibrium. The EM algorithm estimates the haplotype frequency giving consideration to both the allele ratio and the magnitude of the linkage disequilibrium, and accordingly, the EM algorithm is suitable employed for estimating the haplotype frequency in the haplotype estimating method according to the present invention.

In the haplotype estimating method according to the present invention, the haplotype distribution of the population is not directly calculated, but the haplotypes of each individual forming the population are estimated, and the haplotype distribution of the population is calculated based upon the haplotypes of the individuals forming the population. As a consequence, a new graph data structure needs to be formed for handling the genotype data of the individual, i.e., diplotype configuration. Note that the new graph structure for the diplotype configuration can be formed based upon Expression 1.
Kₘ_{ᵢ}ⁿ [Expression 1]

Each individual has a diplotype configuration in which two alleles exist at a locus, and therefore, the graph structure of each individual corresponds to a graph structure in which two alleles are extracted for each locus from the graph structure of the population. Let us say that each individual has genotype (permutation genotype) with a specified phase. In this case, there is a single kind of homozygous permutation genotype, and there are two kinds of heterozygous permutation genotypes. Accordingly, the graph structure for each individual is represented by the n-locus complete graph structure represented by the following Expression 2.
K₂ⁿ [Expression 2]

In this event, edge weighting is made using the diplotype frequency (probability of the diplotype configuration) between the two loci calculated in the population using the EM algorithm instead of the graph structure of the population using the haplotype frequency.

The present method employing the permutation genotype instead of the allele as a node (vertex) can integrally handle the diplotype configuration of each individual. Specifically, the diplotype configuration determined based upon the genotype data of the individual can be represented by the n-node complete graph.

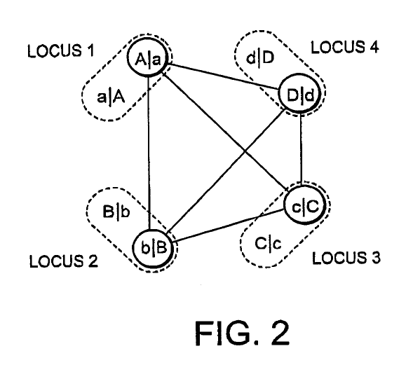

FIG. 2 shows a four-node complete graph structure which represents a four-locus data structure.

With the vertex corresponding to the locus i as vi=(vi1, vi2), the edge between the vertex vi1 corresponding to the locus i and the vertex vj1 corresponding to the locus j is weighted by the joint probability p that the individual has both vi1 and vj1. The n-node complete graph which represents the actual diplotype configuration of the individual is assumed that each side is weighted by a high joint probability. That is, the aforementioned n-node complete graph for the individual is assumed that no edge is weighted by a low joint probability. Giving consideration to the aforementioned fact, the present invention proposes the evaluation value (score) for selection of the vertex set S={v1′, v2′, . . . , vn′} as represented by the following Expression 3.

$$score ( S ) = {\prod }_{i = 1}^{n - 1} {\prod }_{j = i + 1}^{n} p ( {v}_{i}^{′} , {v}_{j}^{′} ) \qquad [ Expression 3 ]$$

It is assumed that the diplotype configuration D(ind) of the individual exhibits the maximum evaluation value, and therefore, the diplotype configuration of the individual can be determined by detecting the n-node complete graph exhibiting the maximum evaluation value based upon the n-locus complete graph. This processing is represented by the following Expression 4.
D(ind)=S′(S′: max score(S)) [Expression 4]

The conventional method in which the entire combination of the vertices of the graph structure is estimated for estimating the diplotype configuration of the individual requires time-calculations of O(2ⁿ) for the number of the loci n, leading to difficulty of estimation, depending upon the number of the loci. Accordingly, there is the need for a new estimating method in which the edge weighting is represented by logarithm, and a n-node complete graph with the maximum sum of edge-weighting is selected from the n-locus complete graph in order to enable high-speed estimation of the haplotypes.

Let us say that vi represents the vertex corresponding to the permutation genotype at the locus i, e(i, j) represents the edge between the vertices corresponding to the locus i and the locus j, and the edge set E is classified into the edge set Ea which is used for determination, and the edge set Ep which is not used for determination. In this case, the edge e(i, j) is weighted by the diplotype frequency, and accordingly, the edges e(i1, j1) and e(i2, j2), and e(i1, j2) and e(i2, j1) are weighted by the same values, respectively. It is noted here that the vertex vi and the edge e(i, j) are represented by the following Expressions 5and 6.
vᵢ={vᵢ₁, vⁱ²} [Expression 5]
eᵢⱼ={eᵢ_{₁}ⱼ_{₁}, eᵢ_{₁}ⱼ_{₂}, eᵢ_{₂}ⱼ_{₁}, eᵢ_{₂}ⱼ₂) [Expression 6]

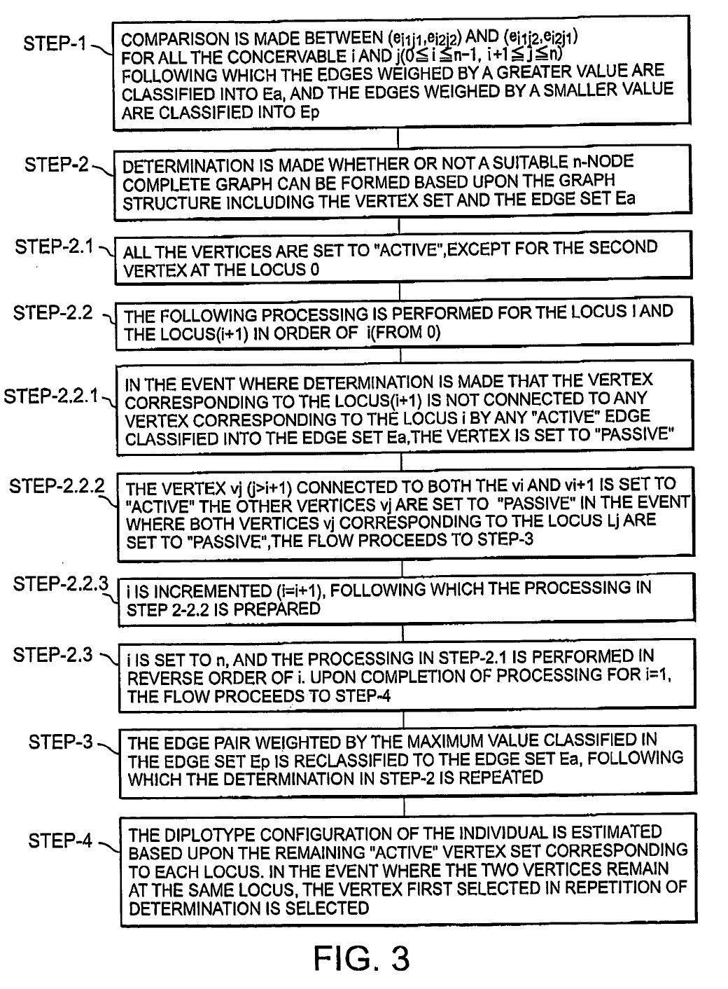

FIG. 3 shows the algorithm for extracting the n-node complete graph structure with the maximum sum of the edge weighting based upon the n-locus complete graph structure represented by the Expression 2 for estimating the diplotype configuration of the individual.

First, comparison is made between e(i1, j1) (=e(i2, j2)) and e(i1, j2) (=e(i2, j1)) for all the conceivable i and j (0≦j≦n−1,i+1≦j≦n). The edges weighted by a greater value are classified into Ea, and the edges weighted by a smaller value are classified into Ep (Step-1).

Subsequently, determination is made whether or not a suitable n-node complete graph can be formed based upon the graph structure including the vertex set and the edge set Ea (Step-2). Specifically, first, all the vertices are set to “active”, except for the second vertex at the locus 0(Step-2.1). Then, the following processing is performed for the locus I and the locus (i+1) in order of i (from 0) (Step-2-2).

In case where determination is made that the vertex corresponding to the locus (i+1) is connected to any vertex corresponding to the locus i by any “active” edge classified into the edge set Ea, the vertex is set to “passive” (Step-2.2.2). Then, the vertex vj (j>i+1) connected to both the vi and vi+1 is set to “active”, otherwise the vertices vj is set to “passive”. If both vertices vj corresponding to the locus Lj are set to “passive”, the flow proceeds to Step-3 (Step-2.2.2). Then, i is incremented (i=i+1), following which the processing in Step S-2.2.2 is repeated (Step-2.2.3).

Next, i is set to n, and the processing in Step-2.1 is performed in reverse order of i. Upon completion of processing for i=1, the flow proceeds to Step-4 (Step-2.3).

Successively, in Step-3, the edge pair weighted by the maximum value classified in the edge set Ep is reclassified to the edge set Ea, following which the determination in Step-2 is repeated (Step-3).

Finally, the diplotype configuration of the individual is estimated based upon the remaining “active” vertex set corresponding to each locus. If the two vertices remain at the same locus, the vertex first selected in repetition of determination is selected (Step-4).

The n-node complete graph estimating algorithm has the advantage of reduced calculation complexity of O(n⁴) and the reduced space complexity of O(n⁴) as compared with the haplotype estimation using the EM algorithm which has the disadvantage of a great deal of calculation complexity and the space complexity of O(2ⁿ).

Description has been made regarding a data structure and algorithm without consideration to missing data of the loci. However, all the genotypes are hardly measured at desired loci, i.e., there are some loci where the genotypes have not been measured. In practice, it is necessary to compensate for this unknown data of the loci.

In the estimation using the EM algorithm, all the conceivable permutation genotypes are estimated for all the missing loci, thereby allowing the researcher to compensate for the missing loci. However, the haplotype estimating method according to the present invention, in which all the conceivable permutation genotypes are estimated, cannot be represented by Expression 2, leading to difficulty in detection of the complete graph structure with the maximum edge weighting due to increased complexity.

However, it is assumed that the estimation results calculated without the missing loci almost always match the estimation results with the missing loci in a case of a sufficiently small number of missing loci as compared with the total number of the loci which are to be estimated. Accordingly, in the haplotype estimating method according to the present invention, first, estimation is made based upon the data without missing loci, following which the data of the missing loci is compensated based upon the estimation results thus obtained.

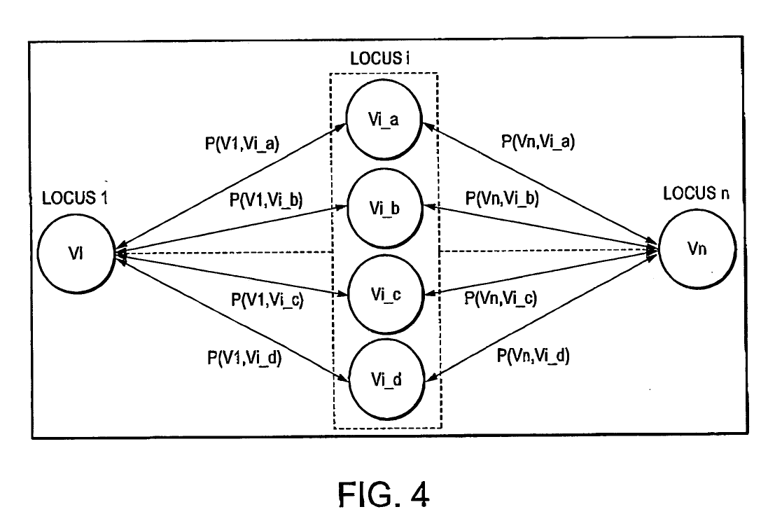

Let us say that estimation is made for the genotype data with a single missing locus of n loci which are to be estimated, for convenience of the following description. In this case, the loci other than the missing locus are estimated using “Idlight” , and the permutation genotypes thereof are determined based upon the estimation results thus obtained. FIG. 4 is a diagram for describing the situation.

In this stage, the permutation genotypes (which are represented by the vertices in the graph) at the loci other than the missing locus i have been determined, and accordingly, the joint probability of the vertex i (which is represented by the edge weighting in the graph) can be calculated by the following Expression 7.
P(V₁,Vᵢ), P(V₂,Vᵢ), . . . , P(Vₙ,Vᵢ) [Expression 8]

In the same way as with the algorithm of the “Idlight” , it is assumed that the most likely estimation results exhibit high joint probability between the missing locus i and the other loci. Specifically, the estimation results exhibiting low joint probability between the missing locus i and any of the other loci does not match the actual haplotypes of the individual with high probability. As described above, the score(vi) which represents the evaluation value for the permutation genotype at the missing locus i is represented by the following Expression 8.
score(Vi)=P(V₁,Vᵢ)×P(V₂,Vᵢ)× . . . ×P(Vₙ,Vᵢ) [Expresion 7]

If the SNP of the conceivable alleles 1 and 2 exists at the missing locus, there are four kinds of the permutation genotypes of (1, 1), (1, 2), (2, 1), and (2, 2). With the four kinds of the permutation genotypes as Vi1, Vi2, Vi3, and Vi4, the vertex which is to be compensated exhibits the maximum value in the above Expression 8, as represented by the following Expression 9.

$${V}_{i} = {max}_{1 \leq j \leq 4} score ( {V}_{ij} ) \qquad [ Expression 9 ]$$

Input data, in which multi-allele exist at the missing locus, may lead to a problem of increased calculations of the score, depending upon the number of the alleles. However, in a case of analyzing the SNP data, estimation is performed for each locus by only four score calculations. It is therefore assumed that the aforementioned compensation of the missing locus is performed without a problem of execution time.

### EXAMPLE 1

The present inventors made analysis for two kinds of data sets of disclosed real data and the simulation data, and made comparison for the precision and execution time between the present analyzing method and the EM algorithm. In particular, comparison was made for the execution time between the present analyzing method and other software for handling haplotype estimation of the multiple loci, Description will be made below regarding the estimation results.

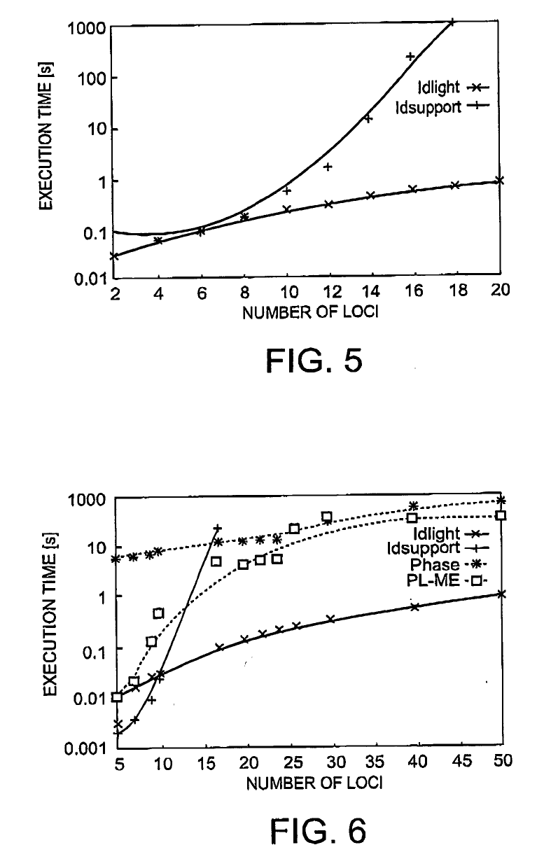

The present inventors measured the execution time using the SNP data regarding the IBD in the 5p31 region of the human chromosome disclosed in Reference 31. FIG. 5 shows the measurement results. Herein, “LDSUPPORT” disclosed in the Reference 22 was used as software using the EM algorithm.

As can be understood from FIG. 5, the EM algorithm exhibits high processing speed for a small number of the loci. However, the haplotype estimating method (idlight) according to the present invention always exhibits shorter executing time than the conventional method (LDSUPPORT) using the EM algorithm. For example, the haplotype estimating method according to the present invention exhibits high processing speed, approximately 1350 times that of the conventional method (LDSUPPORT) for the data of the eighteen loci,

As a comparative examination of the estimation precision, the present inventors made analysis of the SNP data corresponding to the first five blocks of the reported haplotype blocks for 100 individuals. The population was classified into a case population (patient population) and a controlled population (non-patient population), and comparison was made between the haplotype frequency results estimated with the haplotype estimating method according to the present invention and the EM algorithm, for each population. The comparison results are shown in the following Table 1.

TABLE 1

| | |
|---|---|
| case | control |

| | | | | |
|---|---|---|---|---|
| | pro- | EM | pro- | EM |
| haplotype | posal | algorithm | posal | algorithm |

| | | | | | |
|---|---|---|---|---|---|
| block1 | GGACAACC | 0.800 | 0.800 | 0.725 | 0.725 |
| | AATTCGTG | 0.130 | 0.130 | 0.170 | 0.170 |
| | others | 0.070 | 0.070 | 0.105 | 0.105 |
| | total | 1.000 | 1.000 | 1.000 | 1.000 |
| block2 | TTACG | 0.640 | 0.840 | 0.725 | 0.724 |
| | CCCAA | 0.115 | 0.115 | 0.165 | 0.164 |
| | others | 0.045 | 0.045 | 0.110 | 0.112 |
| | total | 1.000 | 1.000 | 1.000 | 1.000 |
| block3 | CGGAGACGA | 0.475 | 0.482 | 0.435 | 0.430 |
| | CGCAGACGA | 0.230 | 0.223 | 0.240 | 0.244 |
| | GACTGGTCG | 0.130 | 0.126 | 0.150 | 0.145 |
| | others | 0.165 | 0.169 | 0.175 | 0.181 |
| | total | 1.000 | 1.000 | 1.000 | 1.000 |
| block4 | CGCGCCCGGAT | 0.505 | 0.500 | 0.415 | 0.412 |
| | CTGCTATAACC | 0.135 | 0.135 | 0.160 | 0.160 |
| | CTGCCCCGGCT | 0.110 | 0.119 | 0.135 | 0.151 |
| | others | 0.250 | 0.246 | 0.290 | 0.277 |
| | total | 1.000 | 1.000 | 1.000 | 1.000 |
| block5 | CCAGC | 0.530 | 0.517 | 0.450 | 0.459 |
| | CCACC | 0.195 | 0.184 | 0.235 | 0.240 |
| | GCGCT | 0.105 | 0.118 | 0.150 | 0.149 |
| | others | 0.170 | 0.181 | 0.165 | 0.152 |
| | total | 1.000 | 1.000 | 1.000 | 1.000 |

The haplotypes with frequency of 10% or more in each block in the population will be referred to as “major haplotypes”, and the others will be referred to as “others”. As can be understood from Table 1, while there is some difference in the block between the present haplotype estimating method and the conventional EM algorithm, it is assumed that the haplotype estimating method according to the present invention achieves estimation with generally the same precision as with the EM algorithm.

Further, the inventors made comparison for the execution time and estimation precision for calculation of the simulation data between the haplotype estimating method according to the present invention and the conventional method. In order to make comparison for the execution time, the present inventors sampled the haplotypes at the 58 loci corresponding to the 3-7 block of the chromosome 5q31 region. Thus, the data was created. Moreover, “PHASE” disclosed in Reference 24 and “PL-EM” disclosed in Reference 25 were employed as comparison data. FIG. 6 shows the comparison results of the execution time with the number of the loci of 5 to 50.

As can be understood from FIG. 6, the EM algorithm having a small overhead executes estimation of the simulation data of the ten or less loci at higher processing speed than with the present haplotype estimating processing. This is because there are a small number of conceivable haplotypes in the data of a small number of the loci, and accordingly, the E-step processing of the EM algorithm is executed at high speed. On the other hand, the present haplotype estimating method, i.e., “Idlight” forms a graph structure with weighting between the loci for each individual, leading to marked loss of execution time due to overhead thereof.

On the other hand, the haplotype estimating method according to the present invention is effectively employed for handling data of 10 or more loci. Specifically, increased loci leads to exponential increase in the conceivable haplotypes, leading to exponential increase in the execution time. However, the haplotype estimating method according to the present invention makes analysis of the graph structure thus formed for each individual, at markedly high speed, thereby suppressing increase in the execution time, and thereby exhibiting markedly high performance for the data of fifteen or more loci as compared with the conventional EM algorithm.

Further, the present Inventors made comparison between the haplotype estimating method according to the present invention, i.e., “idlight” and the conventional method for handling multiple loci as with the “idlight” such as “PHASE” and PL-EM″, and it has been confirmed that the haplotype estimating method according to the present invention achieves almost 100 times as high an execution speed as that of the conventional method, for genotype data of 50 loci. That is, the conventional high-speed estimation method using the EM algorithm has a mechanism for reducing the number of the haplotypes, and accordingly has essentially the same disadvantage as the EM algorithm, On the other hand, the haplotype estimating method according to the present invention determines the haplotype by detecting a suitable complete graph using new data structure, i.e., has an essentially different mechanism.

The haplotype estimating method according to the present invention can handle the data of 100 or more loci at high speed, and can estimate the haplotypes of the genotype data of 100 loci for 100 individuals with execution time of approximately 4 sec, with a 800 MHz Celeron CPU, and with memory requirement of 6.6 Mbyte.

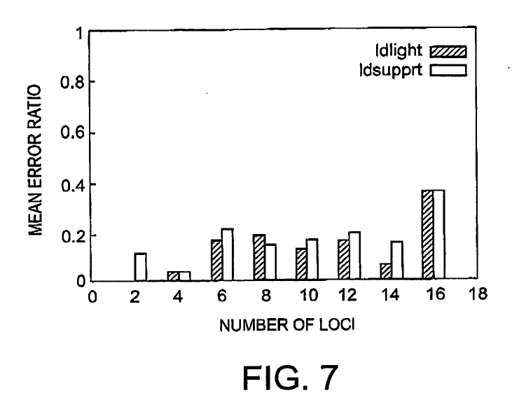

Moreover, the present Inventors made comparison with regard to the estimation precision for the SNP data created by simulation based upon “coalescent with recombination”. FIG. 7, and the following Table 2 shows the average error ratio with the number of individuals fixed at 100, and with the number of loci of from 2 to 16 in increments of 2.

TABLE 2

| Number of | | |
|---|---|---|
| Loci | Idlight | Idsupport |
| 2 | 0.000 | 0.112 |
| 4 | 0.033 | 0.033 |
| 6 | 0.170 | 0.210 |
| 8 | 0.183 | 0.144 |
| 10 | 0.130 | 0.158 |
| 12 | 0.159 | 0.189 |
| 14 | 0.058 | 0.149 |
| 16 | 0.356 | 0.359 |

The average error ratio is the average of the error ratio at the time of analysis of 1000 data sets.

As can be understood from FIG. 7 and Table 2, while the “idlight” exhibits a higher error ratio at the time of estimation of the data of eight loci as compared with the conventional “idsupport”, the “idlight” exhibits higher estimation precision upon at estimation of all the data other than the eight-locus data as compared with the conventional “idsupport”. The “idlight” achieves complete estimation of the data of two loci for all the individuals.

As described above, the haplotype estimating method according to the present invention exhibits generally the same estimation precision as with the EM algorithm for analysis of the 5q31 region. Further, it has been confirmed that the haplotype estimating method according to the present invention can analyze the data of the 14 loci created based upon the coalescent-based model with an error ratio of 0.2 or less. Namely, the haplotype estimating method according to the present invention exhibits the estimation precision with generally the same precision as with the EM algorithm. From the overall perspective, the haplotype estimating method according to the present invention exhibits higher performance than with the EM algorithm. The simulation data used in the above estimation is created using the coalescent-based model based upon the population-genetics theory, and therefore, it is assumed that the haplotype estimating method according to the present invention exhibits generally the same precision for analysis of the real population. Consequently, it has been confirmed that the haplotype estimating method according to the present invention exhibits generally the same estimation precision as with the EM algorithm.

As mentioned above, making comparison with regard to the execution time and estimation precision between the haplotype estimating method according to the present invention and the EM algorithm, it has been confirmed that the haplotype estimating method according to the present invention achieves estimation with greatly reduced execution time while maintaining generally the same precision as with the EM algorithm.

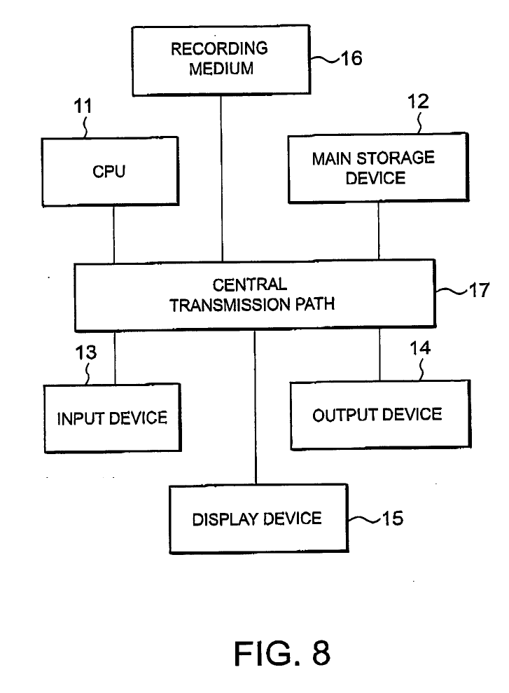

Next, description will be made of a computer system for realizing the haplotype estimation method according to the present invention with Reference to FIG. 8.

As illustrated in FIG. 8, the computer system comprises a CPU 11, a main storage device 12, an input device 13, and output device 14, a display device 15, a recording medium 16, and a central transmission path 17 mutually connecting the above components.

The input device 13 is a device for inputting data, and comprises a keyboard, a pointing device such as a mouse. An example of data to be input is genotype. The output device 14 is a device for outputting the results obtained by the CPU 11 executing the functions thereof, such as a printer. The display device 15 is a device for displaying results the results obtained by the CPU 11 executing the functions thereof ,such as a monitor. The recording medium 16 is a hard disk or the like.

The program for executing the haplotype estimation method according to the above-described present embodiment (hereafter also referred to as “program according to present invention”), and the input data, are saved on the storage medium 16. The CPU 11 receives signals from the input device 13, calls the program according to the present invention up to the main storage device 12, and executes the program.

The CPU 11 calls the input data up to the main storage device 12, and executes the program according to the present invention. The obtained results, i.e., the haplotype estimation results, are displayed on the display device 15, and saved in the recording medium 16. The obtained results may also be output from the output device 14.

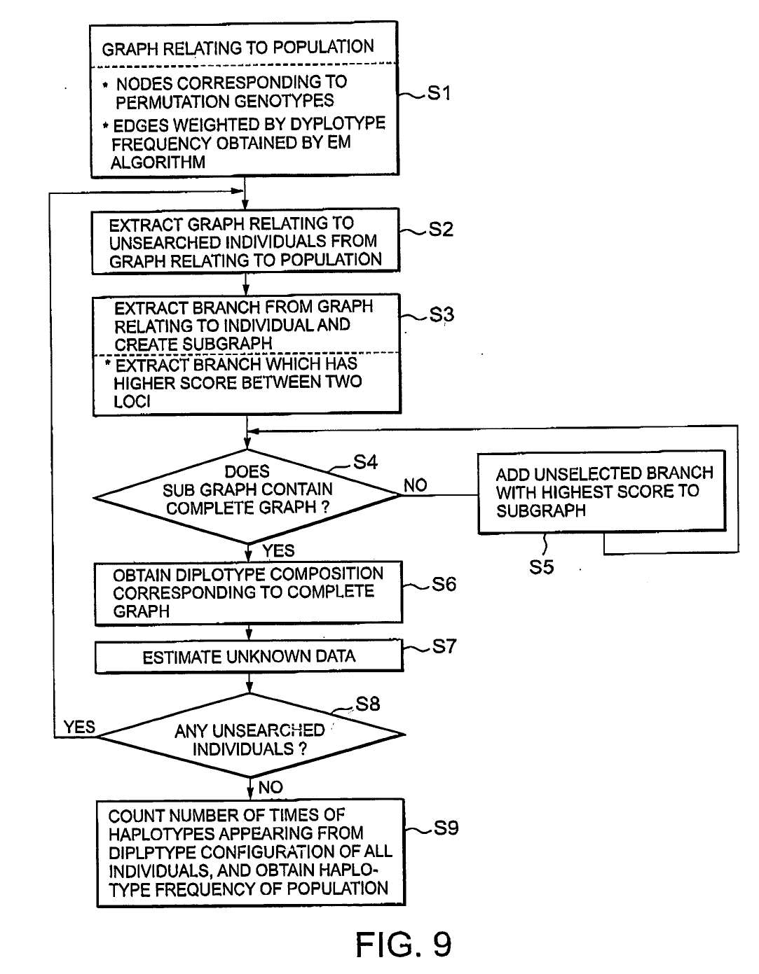

FIG. 9 is a flowchart describing the program according to the present invention.

First, the CPU 11 creates a graph relating to the population (step S1). Specifically, the diplotype configurations of individuals are represented by an n-locus complete graph structure with multiple permutation genotypes corresponding to the vertices (nodes). The edges are weighted by the diplotype frequency (probability of diplotype configuration) obtained by the EM algorithm.

Next, the CPU 11 extracts a graph relating to an unsearched individual from the graph relating to the population (step S2), retrieves a branch from the graph relating to the individual, and creates a subgraph (step S3). The CPU 11 then determines whether the subgraph contains a complete graph (step S4). In case where the subgraph does not contain a complete graph (no in step S4), the CPU 11 adds the unselected branch with the highest score to the graph (step S5), and the flow returns to step S4.

On the other hand, in case where the subgraph contains a complete graph (yes in step S4), the CPU 11 obtains a diplotype configuration corresponding to the complete graph (step S6). The CPU 11 then estimates unknown values (step S7). The CPU 11 determines whether or not there are unsearched individuals (Step S8), and in case where there are unsearched individuals (yes in Step S8), the flow returns to step S2.

In case where there are no unsearched individuals (no in Step S8), the CPU 11 counts the number of occurrences of each haplotype from the diplotypes of all individuals in order to obtain the haplotype frequency of the population (step S9).

More specifically, the haplotype frequency of the population is obtained by estimating the diplotype configuration of each individual in the population, tabulating by haplotype, and obtaining the percentage. For example, considering a case where there are N individuals in a population, and a certain haplotype a exists in the estimated diplotype configurations of the individuals that have been estimated, the haplotype frequency is a/(2N).

Executing the program shown in FIG. 9 with the computer system shown in FIG. 8 realizes the haplotype estimating device according to the present invention, In other words, the haplotype estimating device according to the present invention is a device for estimating haplotypes from genotype data with n loci. The haplotype estimating device comprises: means for representing diplotype configurations for each individual as an n-locus complete graph structure with multiple permutation genotypes corresponding to the vertices; means for taking the weight of each edge of the n-locus complete graph structure as probability of diplotype configurations estimated by maximum-likelihood estimation from the genotype data; means for selecting a set of vertices of the n-locus complete graph structure using a predetermined score; means for estimating diplotype configurations of each individual by solving an n-node complete graph having a maximal score; and means for obtaining haplotype frequency of the population from the diplotype configuration of each individual obtained by the estimation.

In the device for estimating haplotypes, the EM algorithm may be used for maximum-likelihood estimation. Further, the score may represented by the above-described Expression 3, wherein the vertex of a locus i (i being an integer in the range of 1 to n−1) is represented as vi={vi1, vi2}, and an edge connecting the vertex vi1 of the locus i and a vertex vj1 of a locus j (j being an integer in the range of i+1 to n) is weighted by the joint probability p (vi1, vj1) of the individual having the vertex vi1 and the vertex vj1.

In addition, the device may further comprise means for handling individuals with unknown values in the genotype data by estimating the diplotype configuration excluding the unknown values, and then performing complementation using the results, In this case, wherein the score of the permutation genotype for the unknown-value portion may be represented by the above-described Expression 8, wherein each joint probability regarding an unknown locus i is represented as P(V1, Vi), (V2, Vi), . . . (Vn, Vi).

While the present invention has been described by way of preferred embodiments, it should be understood that the present invention is in no way restricted to the embodiments. For example, while the embodiments have been described with Reference to an example wherein the polymorphism markers are SNPs, it is quite evident that one skilled in the art could readily apply other polymorphism markers such as micro-satellite markers with multiple alleles, for example. Also, while description has been made regarding only a case of using the EM algorithm for maximum-likelihood estimation, it is needless to mention that other types of maximum-likelihood estimation may be used as well. Further, the probability of diplotypes which the individual is capable of assuming may be estimated, as well.

1. A method for estimating diplotype configurations of each individual from genotype data with n loci, using a computer system, comprising the steps of:
representing the diplotype configurations for each individual as an n-locus complete graph structure with multiple permutation genotypes corresponding to vertices;
taking a weight of each edge of the n-locus complete graph structure as probability of the diplotype configurations estimated by maximum-likelihood estimation from the genotype data;
selecting a set of vertices of the n-locus complete graph structure using a predetermined score; and
estimating the diplotype configurations of each individual by solving an n-node complete graph having a maximal score.

2. A method for estimating diplotype configurations according to claim 1, wherein:
an EM algorithm is used for the maximum-likelihood estimation.

3. A method for estimating diplotype configurations according to claim 1, wherein:
the score is represented by
$$score ( S ) = {\prod }_{i = 1}^{n - 1} {\prod }_{j = i + 1}^{n} p ( {v}_{i}^{′} , {v}_{j}^{′} )$$
wherein the vertex of a locus i (i being an integer in the range of 1 to n−1) is represented as vi={vi1, vi2}, and an edge connecting the vertex vi1 of the locus i and a vertex vj1 of a locus j a being an integer in the range of i+1 to n) is weighted by a joint probability p (vi1, vj1) of the individual having the vertex vi1 and the vertex vj1.

4. A method for estimating diplotype configurations according to claim 1, further comprising the steps of:
handling individuals with unknown values in the genotype data by estimating the diplotype configuration excluding the unknown values, and;
performing complementation using the results.

5. A method for estimating diplotype configurations according to claim 4, wherein:
the score of the permutation genotype for the unknown-value portion is represented by
score(V₁)=P(V₁,V₁)×P(V₂,Vᵢ)× . . . ×P(Vₙ,Vᵢ)
wherein each joint probability regarding an unknown locus i is represented as P(V₁, V₁), (V₂, Vᵢ), . . . (Vₙ, Vᵢ).

6. A method for estimating haplotypes from genotype data with n loci, using a computer system, comprising the steps of:
representing diplotype configurations for each individual as an n-locus complete graph structure with multiple permutation genotypes corresponding to vertices;
taking a weight of each edge of the n-locus complete graph structure as probability of the diplotype configurations estimated by maximum-likelihood estimation from the genotype data;
selecting a set of vertices of the n-locus complete graph structure using a predetermined score;
estimating the diplotype configurations of each individual by solving an n-node complete graph having a maximal score; and
obtaining haplotype frequency of a population from the diplotype configuration of each individual obtained by the estimation.

7. A method for estimating haplotypes according to claim 6, wherein:
an EM algorithm is used for the maximum-likelihood estimation.

8. A method for estimating haplotypes according to claim 6, wherein:
the score is represented by
$$score ( S ) = {\prod }_{i = 1}^{n - 1} {\prod }_{j = i + 1}^{n} p ( {v}_{i}^{′} , {v}_{j}^{′} )$$
wherein the vertex of a locus i (i being an integer in the range of 1 to n−1) is represented as vi={vi1, vi2}, and an edge connecting the vertex vi1 of the locus i and a vertex vj1 of a locus j a being an integer in the range of i+1 to n) is weighted by a joint probability p (vi1, vj1) of the individual having the vertex vi1 and the vertex vj1.

9. A method for estimating haplotypes according to claim 6, further comprising the steps of:
handling individuals with unknown values in the genotype data by estimating the diplotype configuration excluding the unknown values, and;
performing complementation using the results.

10. A method for estimating haplotypes according to claim 9, wherein:
the score of the permutation genotype for the unknown-value portion is represented by
score(Vi)=P(V₁,Vᵢ)×P(V₂,Vᵢ)× . . . ×P(Vₙ,Vᵢ)
wherein each joint probability regarding an unknown locus i is represented as P(V1, Vi), (V2, Vi), (Vn, Vi).

11. A device for estimating diplotype configurations of each individual from genotype data with n loci, said device, comprising:
means for representing the diplotype configurations for each individual as an n-locus complete graph structure with multiple permutation genotypes corresponding to vertices;
means for taking the weight of each edge of the n-locus complete graph structure as probability of the diplotype configurations estimated by maximum-likelihood estimation from the genotype data;
means for selecting a set of vertices of the n-locus complete graph structure using a predetermined score; and
means for estimating the diplotype configurations of each individual by solving an n-node complete graph having a maximal score.

12. A device for estimating diplotype configurations according to claim 11, wherein:
an EM algorithm is used for maximum-likelihood estimation.

13. A device for estimating diplotype configurations according to claim 11, wherein:
the score is represented by
$$score ( S ) = {\prod }_{i = 1}^{n - 1} {\prod }_{j = i + 1}^{n} p ( {v}_{i}^{′} , {v}_{j}^{′} )$$
wherein the vertex of a locus i (i being an integer in the range of 1 to n−1) is represented as vi={vi1, vi2}, and an edge connecting the vertex vi1 of the locus i and a vertex vj1 of a locus j a being an integer in the range of i+1 to n) is weighted by a joint probability p (vi1, vj1) of the individual having the vertex vi1 and the vertex vj1.

14. A device for estimating diplotype configurations according to claim 1, further comprising:
means for handling individuals with unknown values in the genotype data by estimating said diplotype configuration excluding the unknown values, and then performing complementation using the results.

15. A device for estimating diplotype configurations according to claim 14, wherein:
the score of the permutation genotype for the unknown-value portion is represented by
score(Vi)=P(V₁,Vᵢ)×P(V₂, Vᵢ)× . . . ×P(Vₙ,Vᵢ)
wherein each joint probability regarding an unknown locus i is represented as P(V1, Vi), (V2, Vi), . . . (Vn, Vi).

16. A device for estimating haplotypes from genotype data with n loci, comprising:
means for representing diplotype configurations for each individual as an n-locus complete graph structure with multiple permutation genotypes corresponding to the vertices;
means for taking a weight of each edge of the n-locus complete graph structure as probability of diplotype configurations estimated by maximum-likelihood estimation from the genotype data;
means for selecting a set of vertices of the n-locus complete graph structure using a predetermined score;
means for estimating the diplotype configurations of each individual by solving an n-node complete graph having a maximal score; and
means for obtaining haplotype frequency of a population from the diplotype configuration of each individual obtained by the estimation.

17. A device for estimating haplotypes according to claim 16, wherein:
an EM algorithm is used for the maximum-likelihood estimation.

18. A device for estimating haplotypes according to claim 16, wherein:
the score is represented by
$$score ( S ) = {\prod }_{i = 1}^{n - 1} {\prod }_{j = i + 1}^{n} p ( {v}_{i}^{′} , {v}_{j}^{′} )$$
wherein the vertex of a locus i (i being an integer in the range of 1 to n−1) is represented as vi={vi1, vi2}, and an edge connecting the vertex vi1 of the locus i and a vertex vj1 of a locus j (0 being an integer in the range of i+1 to n) is weighted by a joint probability p (vi1, vj1) of the individual having the vertex vi1 and the vertex vj1.

19. A device for estimating haplotypes according to claim 16, further comprising:
means for handling individuals with unknown values in the genotype data by estimating the diplotype configuration excluding the unknown values, and then performing complementation using the results.

20. A device for estimating haplotypes according to claim 19, wherein:
the score of the permutation genotype for the unknown-value portion is represented by
score(Vᵢ)=P(V₁,Vᵢ)×P(V₂,Vᵢ)× . . . ×P(Vₙ,Vᵢ)
wherein each joint probability regarding an unknown locus i is represented as P(V1, Vi), (V2, Vi), . . . (Vn, Vi).

21. A program for causing a computer to estimate diplotype configurations of each individual from genotype data with n loci, the program comprising:
a function for representing the diplotype configurations for each individual as an n-locus complete graph structure with multiple permutation genotypes corresponding to vertices;
a function for taking a weight of each edge of said n-locus complete graph structure as probability of the diplotype configurations estimated by maximum-likelihood estimation from the genotype data;
a function for selecting a set of vertices of the n-locus complete graph structure using a predetermined score; and
a function for estimating the diplotype configurations of each individual by solving an n-node complete graph having a maximal score.

22. A program according to claim 21, wherein:
an EM algorithm is used for the maximum-likelihood estimation.

23. A program according to claim 21, wherein:
the score is represented by
$$score ( S ) = {\prod }_{i = 1}^{n - 1} {\prod }_{j = i + 1}^{n} p ( {v}_{i}^{′} , {v}_{j}^{′} )$$
wherein the vertex of a locus i (i being an integer in the range of 1 to n−1) is represented as vi={vi1, vi2}, and an edge connecting the vertex vi1 of said locus i and a vertex vj1 of a locus j (j being an integer in the range of i+1 to n) is weighted by a joint probability p (vi1, vj1) of the individual having the vertex vi1 and the vertex vj1.

24. A program according to claim 21, further comprising a function for causing the computer to handle individuals with unknown values in the genotype data by estimating the diplotype configuration excluding the unknown values, and then performing complementation using the results.

25. A program according to claim 24, wherein:
the score of the permutation genotype for said unknown-value portion is represented by
score(Vᵢ)=P(V₁, Vᵢ)×P(V₂,Vᵢ)× . . . ×P(Vₙ, Vᵢ)
wherein each joint probability regarding an unknown locus i is represented as P(V1, Vi), (V2, Vi), . . . (Vn, Vi).

26. A program for causing a computer to estimate haplotypes from genotype data with n loci, the program comprising:
a function for representing diplotype configurations for each individual as an n-locus complete graph structure with multiple permutation genotypes corresponding to vertices;
a function for taking a weight of each edge of the n-locus complete graph structure as probability of the diplotype configurations estimated by maximum-likelihood estimation from the genotype data;
a function for selecting a set of vertices of the n-locus complete graph structure using a predetermined score;
a function for estimating the diplotype configurations of each individual by solving an n-node complete graph having a maximal score; and
a function for obtaining haplotype frequency of a population from the diplotype configuration of each individual obtained by the estimation.

27. A program according to claim 26, wherein:
an EM algorithm is used for the maximum-likelihood estimation.

28. A program according to claim 26, wherein:
the score is represented by
$$score ( S ) = {\prod }_{i = 1}^{n - 1} {\prod }_{j = i + 1}^{n} p ( {v}_{i}^{′} , {v}_{j}^{′} )$$
wherein the vertex of a locus i (i being an integer in the range of 1 to n−1) is represented as vi={vi1, vi2}, and an edge connecting the vertex vi1 of said locus i and a vertex vj1 of a locus j a being an integer in the range of i+1 to n) is weighted by a joint probability p (vi1, vj1) of the individual having the vertex vi1 and the vertex vj1,

29. A program according to claim 26, further comprising a function for causing the computer to handle individuals with unknown values in the genotype data by estimating the diplotype configuration excluding the unknown values, and then performing complementation using the results.

30. A program according to claim 29, wherein:
the score of the permutation genotype for the unknown-value portion is represented by
score(Vᵢ)=P(V₁, Vᵢ)×P(V₂,Vᵢ)× . . . ×P(Vₙ,Vᵢ)
wherein each joint probability regarding an unknown locus i is represented as P(V1, Vi), (V2, Vi), . . . (Vn, Vi).
